(19) United States
(12) Patent Application Publication
(10) Pub. No.: US 2003/0143757 A1
(43) Pub. Date: Jul. 31, 2003

(54) Methods for identifying drug cores

(76) Inventors: Jonathan Moore, Carlisle, MA (US); Guy William Bemis, Arlington, MA (US); Christopher A. Lepre, Somerville, MA (US); Jasna Fejzo, Arlington, MA (US); Jeffrey Weilee Peng, Boston, MA (US); Keith Phillip Wilson, Brookline, MA (US); Mark Andrew Murcko, Holliston, MA (US)

Correspondence Address: FISH & NEAVE, 1251 AVENUE OF THE AMERICAS, 50TH FLOOR, NEW YORK, NY, 10020-1105, US

(21) Appl. No.: 10/281,471

(22) Filed: Oct. 25, 2002

Related U.S. Application Data
(63) Continuation of application No. 09/621,910, filed on Jul. 24, 2000, now abandoned.
Application No. 09/621,910 is a continuation of application No. 09/022,022, filed on Feb. 11, 1998, now abandoned.
(60) Provisional application No. 60/050,060, filed on Jun. 13, 1997.

(51) Int. Cl. G01N 33/543; G06F 19/00; G01N 33/48; G01N 33/50

(52) U.S. Cl. 436/518; 702/19

(57) ABSTRACT

The present invention relates to methods for detecting chemical moieties that may serve as the core or scaffold of a potential drug that is directed to a target. The invention further relates to a chemical library of drug cores and the use of that library to identify useful drug cores for a particular target protein.

### CROSS-REFERENCE TO PRIOR APPLICATION

This application claims the benefit of U.S. Provisional Application No. 60/050,060, filed Jun. 13, 1997.

### TECHNICAL FIELD OF THE INVENTION

The present invention relates to methods for detecting chemical moieties that may serve as the core or scaffold of a potential drug that is directed to a target. The invention further relates to a chemical library of drug cores and the use of that library to identify useful drug cores for a particular target protein.

### BACKGROUND OF THE INVENTION

In a target directed drug discovery program, there are many different strategies that may used to identify a clinical candidate. Although these different approaches to drug discovery might follow significantly different pathways of optimization to a highly potent and bioavailable drug molecule, they all share a common origin: they must begin with a lead compound. Many of the properties of the final compound or class of compounds, e.g. target affinity, inhibitory potential, solubility, and bioavailability, may be inextricably tied to those of the initial lead compound. Therefore, the methods by which leads are identified in the early stages could significantly impact the success of the project in the latter stages.

Lead molecules are currently identified and selected in a number of different ways. A lead molecule may be a known drug molecule, or an analog of a known drug. Alternatively, if the target is an enzyme, the lead may be a substrate or substrate analog. Often, a lead is discovered by random screening of either commercially available or proprietary compound libraries, or both. However, there are many potential problems which may arise using the above strategies. For example, starting with a competitor's drug may not lead to sufficient diversity in the final class of compounds to avoid impinging upon that competitor's intellectual property.

Starting with a bioactive natural product may necessarily force the design of large molecular weight analogs, with poor synthetic accessibility and difficult scale-up problems. Alternatively, using random screening to generate leads might result in a novel, synthetically accessible class of compounds, but unless several multiple hits from random screening are optimized in parallel, the compound class might lack sufficient diversity to overcome problems related to solubility or bioavailability without overly compromising potency.

When random screening is used to generate leads in a drug design program, there are several approaches which may be taken. A brute force approach is to screen very large (>100,000) numbers of compounds, identify a potent binder or inhibitor of the drug target, and then modify that binder to optimize its activity against the drug.

In a structure-based program, a more rational approach is to start by using information-driven methods for virtual screening of databases to select a smaller subset of compounds for high throughput screening. This approach requires that structural information about the target and particularly its binding site(s) be available.

Computer modeling is then routinely employed to search available databases for good screening candidates. Then, a subset of representative compounds is assayed for binding/inhibition. Weak binders/inhibitors then become leads for interative structure based drug design or SAR, depending on what structural information regarding the target is available.

The downside of this approach is that in choosing representative compounds for actual binding/inhibition studies one may miss potentially important leads. This may occur because too small a number of representative compounds were chosen for screening or that there was insufficient diversity in that representative set.

Thus, there is still a need for improved methods of identifying lead compounds. Moreover, it would be particularly advantageous if the generation of lead compounds could be efficiently and accurately achieved without the need for obtaining complex structural information about the target.

Another important aspect of lead generation is the ability to detect compounds that bind weakly to the target. Such compounds include those that bind to the active site of an enzyme, but do not inhibit its enzymatic function. Lead compounds such as these go undetected in enzyme inhibition assays. Moreover, many of the standard assays used to detect binding have a limit of detection in the micromolar range. However, many lead compounds that bind in the millimolar range may have desirable properties (e.g., ease of synthesis, good solubility, good bioavailability) and can ultimately be optimized to significantly increase binding.

The use of one type of NMR technology to design and identify weakly binding compounds is disclosed in PCT Publication Nos. WO 97/18471 and WO 97/18469. The NMR technology disclosed in these documents, however, suffers from several shortcomings. First, it can be only be applied to targets of low molecular weight (<20 kDa). Second, it requires isotopic labeling of the target which is both expensive and usually results in lower yields of proteins. Third, and most important, that technique requires that the x-ray crystal or NMR structure of the target be solved prior to employing the method.

Thus, there is still a need for techniques which can detect weak binding of ligands to targets which are either large in size and/or for which no structural information is available.

### SUMMARY OF THE INVENTION

The present invention solves the problems indicated above by providing a method for detecting weak binding of potential drug cores to targets, regardless of the size of the target and without requiring x-ray crystallographic or NMR structural information about the target.

The method of this invention preferably incorporates the use of NMR to detect binding, particularly the techniques known as transferred nuclear Overhauser effect (“tNOE”), differential line broadening (“DLB”), relaxation filtering, and pulsed-field-gradient NMR spectroscopy (“PFG NMR”).

In this method, a single compound selected from a chemical library of known drug cores or a mixture of such compounds is combined with a target and subjected to NMR. The ligand or ligands in this mixture which bind weakly to the target bind to and come off the target numerous times during the NMR procedure. NOEs built up by these ligands in the bound state are transferred to the ¹H NMR signals of excess free ligand. As a result, the relative signs of diagonal and cross peaks in the spectrum changes with respect to those observed for the free ligand alone, thus providing an unambiguous indication of binding.

Similarly this rapid equilibrium between the bound and the unbound state creates a characteristic decrease in amplitude of peak heights and, in most cases, a broadening of one or more peaks in the one-dimensional NMR spectrum of the ligand. This, too, can provide an unambiguous indication of binding in the methods of this invention.

In PFG NMR, the diffusion coefficient of the drug core alone and in the presence of the target are compared. If the core binds to the target, than the detected diffusion coefficient of the drug core is reduced. The amount by which that coefficient is reduced can be used to calculate a K_{d} value.

Once identified as being capable of binding to the target, these drug cores can be modified and optimized by the addition of side groups to create a potential drug candidate. In addition, two or more drug cores that display binding to the target can be combined into a single molecule to optimize and increase binding affinity.

The invention also provides a relatively small library of soluble carbocyclic and heterocyclic ring systems which represent frameworks most commonly found in known drug molecules. Some of these frameworks have been described by G. W. Bemis et al., J. Med. Chem., 39, pp. 2887-2893 (1996). These rings optionally contain one or more of a small group of side chains which are also commonly present in commercially available drugs. The advantage of this library is its small size and its heavy bias towards being “druglike.”

The term “druglike”, as used herein, means properties that are considered important for commercial drugs. These include solubility, bioavailability, ease and low cost of synthesis (including low cost of starting materials and the ability to produce the final product using few synthetic steps), low toxicity, and chemical and metabolic stability.

Because the library of this invention consists of cores and side chains that are present in commercially available drugs, any weak binders detected therein will necessarily have desirable druglike qualities. Moreover, the small size of the library makes screening less time and labor intensive. Finally, the diverse nature of the cores and side chains in the library means that multiple “hits” (i.e., weak binders) are likely, allowing the flexibility and advantages of pursuing several compound classes at once.

Once one or more members of the library are identified as weakly binding a given target, each binder is used to bias the clustering of large chemical databases (either commercially available or proprietary) and to select a group of compounds for high throughput screening.

### BRIEF DESCRIPTION OF THE DRAWINGS

FIG. 1, panel A, depicts the one-dimensional proton NMR spectra of a mixture of nicotinic acid (peaks indicated by an ‘X’) and 2-phenoxy benzoic acid (peaks indicated by a ‘Y’) in the presence of the target, p38. FIG. 1, panel B, depicts the one-dimensional proton NMR spectra of a mixture of nicotinic acid and 2-phenoxy benzoic acid in the absence of target. A relaxation filter was used after the preparatory delay to attenuate broad resonances arising from the protein.

FIG. 2, panel A, depicts the 2D NOESY spectra of a mixture of nicotinic acid (‘X’) and 2-phenoxy benzoic acid (‘Y’) in the absence of target. FIG. 2, panel B, depicts the 2D NOESY spectra of a mixture of nicotinic acid and 2-phenoxy benzoic acid in the presence of p38 MAP kinase.

FIG. 3, panel A, depicts the ω₂ cross-sections from the NOESY spectra shown in FIG. 2. of the mixture of nicotinic acid (‘X’) and 2-phenoxy benzoic acid (‘Y’) in the absence of target. FIG. 3, panel B, depicts the ω₂ cross-sections from the NOESY spectra shown in FIG. 2. of the mixture of nicotinic acid (‘X’) and 2-phenoxy benzoic acid (‘Y’) in the presence of p38 MAP kinase.

FIG. 4 depicts the Water-sLED pulseq sequence for measuring translational diffusion coefficients. Proton 90° rf pulses and solvent flip-back pulses are indicated on the top staff by the black vertical bars and unfilled domes, respectively. Gradients are given on the lower staff and are applied along the Z-axis. Only the phase-encoding and phase-decoding gradients are shaded. The gradients are of length δ=4 ms, and their strengths are identical. The strengths are varied parametrically in a series of one-dimensional experiments. “Δ” indicates the total time between the two gradients. During the “T” period (50 ms), phase-encoded magnetization is aligned with the external magnetic field.

FIG. 5 depicts an example of signal decay in the water-sLED experiment. Gradient strength increases from left to right.

FIG. 6 depicts fits of the peak integrals versus K²=γ²δ²G_{z}²(Δ−δ/3) to determine the diffusion coefficients. Peak integrals for free 2-phenoxybenzoic acid, 2-phenoxybenzoic acid in the presence of p38, and the p38 aromatics, correspond to the open squares, filled diamonds, and filled circles, respectively.

### DETAILED DESCRIPTION OF THE INVENTION

Based upon a survey of molecular shapes which represent frameworks most commonly found in known drug molecules, applicants have designed a library of what we refer to as “drug cores.” Without being bound by theory, we believe that the prevalence of these drug cores in known drug molecules is due, at least in part, to the fact that they impart desirable properties to a drug molecule. These properties include solubility, bioavailability, lack of toxicity, etc.

We believe that a drug design effort that begins by identifying a drug core within our small, but diverse library of low molecular weight, soluble drug cores that binds to a desired target and then builds off of that target (e.g., by adding additional substituents) has a better likelihood in producing a safe and effective drug than other techniques, such as screening thousands of members of a combinatorial library.

The problem that one faces in identifying a drug core that binds a target is that, at best, the cores bind the target with a K_{d} in the μM to mM range. At that binding affinity most of these cores would be missed in a standard enzymological inhibition assay. Applicants have discovered a way around this problem by applying the well known nuclear magnetic resonance (“NMR”) techniques of differential line broadening (“DLB”), relaxation filtering, the transferred nuclear Overhauser enhancement (“tNOE”) and pulsed-field gradient NMR (“PFG”). These techniques may be applied with no limitation on the size of the target and no requirement for isotope labeling of the target to detect the binding of drug cores. Thus, according to one embodiment, the invention provides a method of identifying a drug core suitable for a given target comprising the steps of:

a) providing a drug core consisting of a cyclic structure selected from:

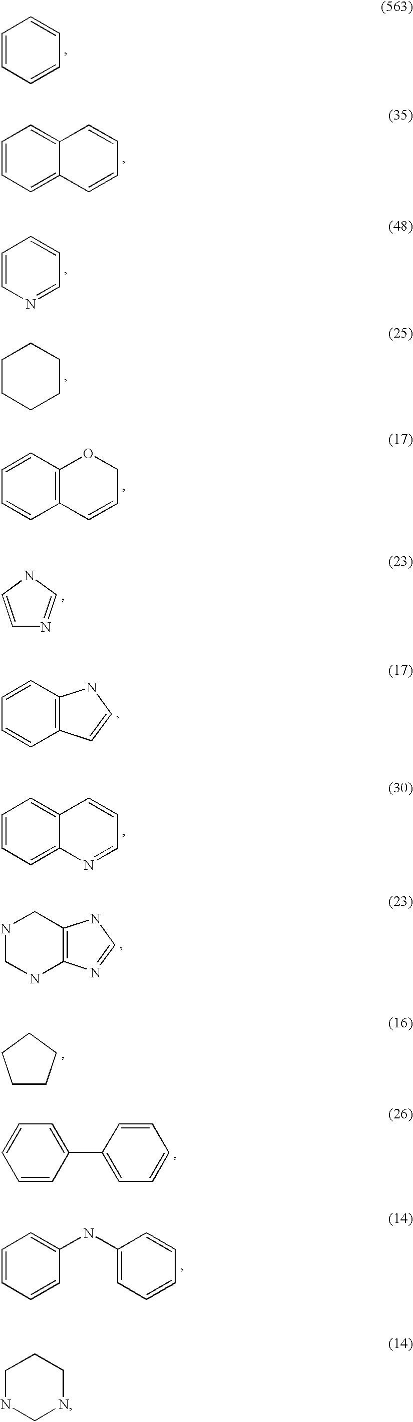

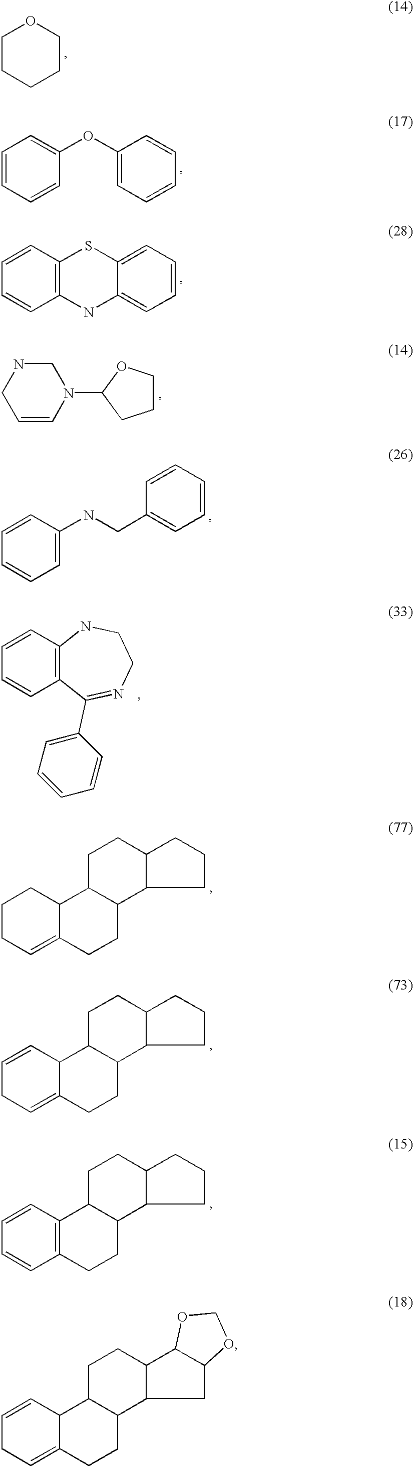

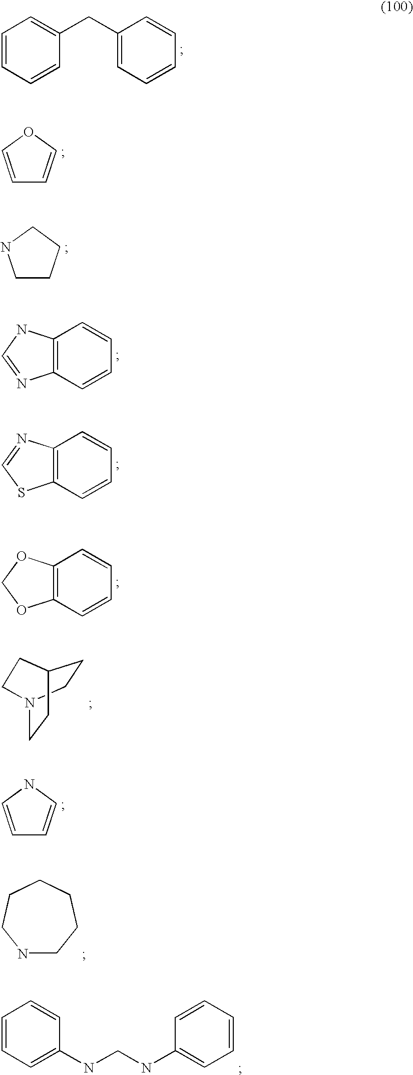

or tautomers thereof, wherein said cyclic structure is optionally substituted at:

i) one or more carbon atoms with one or more substituents independently selected from ═O, —CH₃, —OH, —OCH₃, —Cl, —NH₂, —C(O)OH, —F, —CH₂OH, —CH₂CH₃, —OC(O)CH₃, —NO₂, —N(CH₃)₂, —CF₃, —C(O)NH₂, —C(O)OCH₃, —C(O)OCH₂CH₃, —CH(CH₃)₂, —S(O)₂NH₂, —C(O)CH₃, —CN, —Br, —I, —S(O)₂OH, —OCH₂CH₃, —CH₂C(O)OH, —OC(O)CH₂CH₃, —CH₂CH(CH₃)₂, —C(O)CH₂OH, —N(H)C(O)CH₃, —C(CH₃)₃, ═S, —CH₂NH₂, -OCH₂CH(OH)CH₂N(H)C(CH₃)₃, —N(H)CH₃, —CH(CH₃)C(O)OH, —C≡CH, —(CH₂)₂CH₃, —CH₂C(O)NH₂, —OCH₂CH(OH)CH₂N(H)CH(CH₃)₂, ═N—OCH₃, or —OCH₂CH₃

ii) one or more nitrogen atoms, if present, with a substituent independently selected from —CH₃, —(CH₂)₂OH or —CH₂CH₃;

iii) a sulfur atom, if present, with ═O; and

b) determining whether any one of said drug cores binds to said target.

The term “target”, as used herein, refers to any biologically important molecule which is capable of binding to another molecule. The term includes proteins, particularly enzymes, peptides, nucleic acids, such as DNA and RNA, membrane proteins in detergent or micelles, subcellular structures or organelles, anv of the foregoing attached or tethered to a solid support, or any of the foregoing already bound to a ligand.

A molecule that is already bound to a ligand (e.g., one of the drug cores of this invention) is expected to have sufficient room in its binding pocket to bind additional ligands, such as a second drug core of this invention. In this manner, multiple drug cores that display binding to the target molecule can be combined (and subsequently modified through the addition of substituents) to create a potential drug.

The solvent into which the protein and target are mixed can be any solvent in which both the target and drug core are soluble and stable and which is compatible with NMR or other techniques useful to detect binding. Most preferably, the solvent is an aqueous buffer system.

The choice of drug core will depend upon what, if any, structural information one has about the target. If some structural information is available about the shape or nature of the binding site, one will select those drug cores that have the requisite shape, size and nature to theoretically fit into and interact with that binding site.

If nothing is known about the target, the choice of drug core should be made simply by prevalence of that core in known drugs. The numbers in parentheses below certain of the drug cores depicted above reflects the number of commercially available drugs listed in the Comprehensive Medicinal Chemistry (“CMC”) database (version 94.1) that contain that core. These numbers exclude those compounds which are radiopaque agents, contrast agents, solvents, anesthetics, disinfectants, topicals, local agents, spermicides, wetting agents, flavoring agents, pharmaceutical aids, surgical aids, dental compounds, surfactants, sunscreens, ultraviolet screens, emetics, preservatives, aerosol propellants, chelators, keratolytics, insecticides, astringents, herbicides, laxatives, sweeteners, dental caries prophylactics, adhesives, veterinary compounds, buffers, scabicides and ectoparasiticides. Thus, one would start with the drug core having the highest number in parentheses and, in order, work their way down to the cores that are least prevalent, followed by those cores that do not contain any parenthetical numbers.

More preferably, the cyclic portion of the drug core is a drug core that is prevalent in known drugs and is selected from:

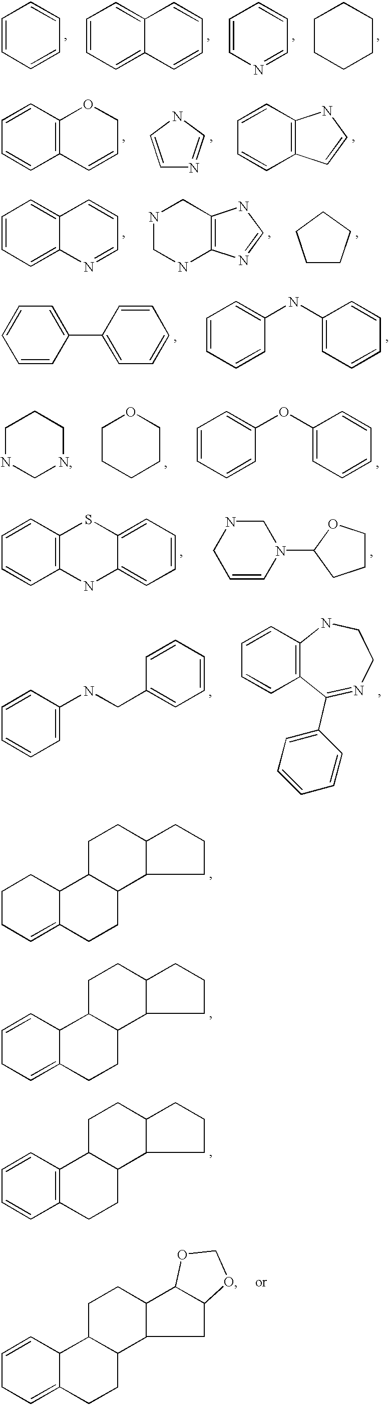

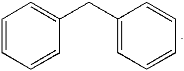

The choice of substituents to place on the drug core is dependent upon two factors: prevalence of those side chains in existing drugs, and the solubility of the drug core in the absence of substituents. In other words, the substituent should be chosen so that the resulting drug core is soluble in the solvent system being used. Those of skill in the art will recognize which of the cyclic structures depicted above will be insoluble in a given solvent system and which substituents will impart increased solubility to that cyclic structure.

More preferably, the optional substituents attached to a carbon atom are independently selected from —CH₃, —OH, —OCH₃, —Cl, —NH₂, —C(O)OH, —F, —CH₂OH, —CH₂CH₃, —OC(O)CH₃, —NO₂, —N(CH₃)₂, —CF₃, —C(O)NH₂, —C(O)OCH₃, —C(O)OCH₂CH₃, —CH(CH₃)₂, —S(O)₂NH₂, —C(O)CH₃, —CN, —Br, —I, —S(O)₂OH, —OCH₂CH₃, —CH₂C(O)OH, —OC(O)CH₂CH₃, —CH₂CH(CH₃)₂, —C(O)CH₂OH, —N(H)C(O)CH₃, —C(CH₃)₃, ═S, —CH₂NH₂, —OCH₂CH(OH)CH₂N(H)C(CH₃)3, —N(H)CH₃, —CH(CH₃)C(O)OH, —C=—CH, —(CH₂)₂CH₃, —CH₂C(O)NH₂, —OCH₂CH(OH)CH₂N(H)CH(CH₃)2 or ═N—OCH₃

Most preferably, the substituents attached to a carbon atom are independently selected from ═O, —OCH₃, —OH, —NH₂, —C(O)OH, —S(O)₂OH, —S(O)₂NH₂, —CH₂OH or —C(O)NH₂; the substituent attached to a nitrogen atom is CH₃; and the substituent attached to a sulfur atom is ═O. These most preferred substituents are both widely prevalent in drugs and impart solubility in aqueous buffers to the ring system to which they are attached.

While the invention envisions that the drug cores can contain any number of substituents that is chemically feasible, it is preferred that the number of substituents be from 0 to 3.

The final step of the method involves determining whether the drug core binds to said target. It is believed that the practical limit of detection of binding requires a K_{d} of less than about 10 millimolar.

As a practical matter, the drug cores listed above are more soluble in organic solvents than in aqueous solution. Thus, they are stored in an organic solvent, such as DMSO, prior to mixing with the target. The target is typically stored in solid form or in an aqueous solution. When the drug core is mixed with the target, the drug core solvent and the target solvent are usually miscible. Because the volume of solvent containing the drug core is far less than the volume of aqueous solution containing the target, even when the two solvents are not miscible, the drug core will transfer from the organic phase to the aqueous phase, thus allowing both the drug core and the target to reside in the aqueous phase.

As set forth above, it is preferred that NMR techniques be used to detect the weak binding of the drug cores to the target. Other, less preferred, methods of detecting such binding include functional activity assays, immunoreactivity, radiological assays such as scintillation proximity and competition with radioactive tracers, spectral methods such as ultra-violet, visible, infrared and fluorescence spectroscopy, circular dichroism, surface plasmon resonance, calorimetry, mass spectrometry, liquid chromatography and equilibrium dialysis.

According to one preferred embodiment, the determination of binding is achieved by the NMR method of line broadening, relaxation filtering or a combination of the two and comprises the steps of:

i) obtaining a one-dimensional NMR spectrum of said drug core in the absence of said target;

ii) mixing the target with the drug core at a molar ratio of between 1:1 and 1:100.

iii) subjecting said mixture to nuclear magnetic resonance for a period of time sufficient to obtain a one-dimensional spectrum; and

iv) comparing the spectra obtained in steps i) and iii) to determine if said drug core has bound to said target.

If binding between the drug core and target has occurred, the width of one or more of the drug core peaks in the drug core +target spectrum will increase as compared to the drug core alone spectrum. Similarly, the amplitude of one or more of the peaks corresponding to a binding drug core will decrease. Preferably, the two methods are used in conjunction wherein the sample is first subjected to a relaxation filter when generating the one-dimensional spectra. This serves to filter out the resonances of the target in the drug core+target spectrum and makes interpretation of line broadening easier. Also, with drug cores that bind in the millimolar range, line broadening may be minimal and not easily detectable. such weak binding drug cores will, however, demonstrate a more easily detectable decrease in peak amplitude caused by the relaxation filter.

An advantage of this technique is that multiple drug cores can be tested in the same sample. In testing multiple drug cores in the same sample, it is preferred that at least one peak in each drug core spectrum be non-overlapping with all of the other peaks from the other drug cores in a one-dimensional spectra. This is so, because when one observes line broadening, one must be able to identify which drug core corresponds to the broadened peak. However, even if all peaks of a particular drug core are overlapped by peaks of other drug cores in the sample, one can still detect binding using subtraction methods. These methods involve subtracting the spectrum obtained from the mixture of drug cores in the absence of target from the spectrum obtained in the presence of the target. Peaks corresponding to drug cores that bind to the target will be visible after the subtraction, while non-binding drug core peaks will be obliterated.

Moreover, when one tests multiple drug cores in a single sample, one should obtain a reference spectrum of the combination of drug cores in the absence of the target, as well as reference spectra for each individual drug core.

The other requirements are that 2 or more of the drug cores in said sample do not interact chemically with one another, with the NMR solvent system, or with the NMR buffer components utilized to determine binding. This is important because the structure of the reacting drug cores will be altered and will not reflect the structure that one desires to test for binding. Also, the products of the reaction may have different NMR spectra, therefore making interpretation of line broadening difficult or impossible. Those of skill in the art will know, based upon drug core structure and buffer conditions to be used in NMR whether 2 or more drug cores would be expected to react with one another.

Lack of interaction also means that the drug cores in the sample should not aggregate or induce precipitation with one another or the target, nor bind covalently to one another. These adverse events will increase the perceived molecular weight of one or more drug cores or the target in the sample or remove them from solution.

Thus according to another embodiment, the determination of binding is performed on a sample containing multiple drug cores mixed with a single target and is achieved by the NMR method of line broadening comprising the steps of:

i) obtaining one-dimensional NMR spectra for each of said drug cores to be tested for binding to said target, wherein said each of said spectra is obtained in the absence of said target

ii) mixing together between 2 and 20 of said drug cores which will not interact with one another;

iii) obtaining a one-dimensional NMR spectrum of said mixture of said drug cores;

iv) mixing said drug cores with the target, wherein each of said drug cores is present at a molar ratio to said target of between 1:1 and 100:1;

v) subjecting said mixture of drug cores and said target to nuclear magnetic resonance for a period of time sufficient to obtain a one-dimensional spectrum; and

vi) comparing the spectra obtained in steps iii) and v) to determine which, if any, of said drug cores has bound to said target.

Although a ratio of target:drug core of 1:100 is envisioned in the line broadening method of detecting binding, it is preferred that the ratio be between 1:10 and 1:1.

According to another preferred embodiment, the determination of binding is achieved by the NMR method of tNOE and comprises the steps of:

i) mixing the target with the drug core at a molar ratio of between 1:1 and 1:100.

ii) subjecting said mixture to nuclear magnetic resonance for a period of time sufficient to obtain a two-dimensional spectrum; and

iv) analyzing the spectra obtained in step ii) to determine if said drug core has bound to said target.

This method may also be used in conjunction with a relaxation filter.

If binding between the drug core and target has occurred, it will induce a change in the NOE of the cross peaks corresponding to the drug core. This will make the sign of the cross peaks the same as the sign of the diagonal peaks. Unbound cores produce a two-dimensional spectrum wherein the signs of the diagonal peaks are usually opposite those of the cross peaks. In those situations where the sign of the unbound drug core is the same as that of the target (i.e., very strong magnetic field or high molecular weight drug core) the amplitude of those peaks will increase significantly if that drug core binds to the target.

The tNOE method may also be utilized with multiple drug cores in a single sample. To do so requires that a one-dimensional NMR spectrum of each individual drug core be obtained, as well as a one-dimensional spectrum of the mixture of drug cores in the absence of the target. The chemical shifts of peaks in the one-dimensional spectra correspond to the chemical shifts of the diagonal peaks observed in a two-dimensional spectrum. Thus, the reference one-dimensional spectra will allow assignment of individual diagonal peaks to a specific drug core. The cross peaks for each individual drug core are then easily identifiable as they appear at the same frequencies as any two diagonal peaks corresponding to that drug core.

Although a ratio of drug core:target of 100:1 is envisioned in the tNOE method of detecting binding, it is preferred that the ratio be between 50:1 and 1:1.

According to yet another embodiment, the determination of binding is achieved using the NMR technique of pulsed field gradients and comprises the steps of:

i) determining a gradient strength that is effective to substantially reduce or eliminate the one-dimensional NMR spectrum of said drug core in the absence of said target;

ii) mixing the target with the drug core at a molar ratio of between 1:1 and 1:20.

iii) subjecting said mixture to nuclear magnetic resonance for a period of time sufficient to obtain one-dimensional spectra using the gradient strength determined in step i); and

iv) analyzing the spectrum obtained in step iii), and, if necessary, comparing said spectrum to a one-dimensional spectrum of said target in the absence of said drug core at the gradient determined in step i), to determine if said drug core has bound to said target.

Although a target:drug core ratio of up to 1:20 is envisioned in all of the PFG NMR methods set forth herein, it is preferable that the ratio be between 1:5 and 1:1. Most preferably, the ratio is about 1:1.

The free drug core will demonstrate a substantially reduced spectrum (i.e., peaks with very little amplitude) or no spectrum at all at the gradient strength utilized. If, however, the drug core has bound to the protein, the effect of the gradient on reducing or eliminating the drug core's spectrum is diminished, and the drug core exhibits a characteristic spectrum. This spectrum is, of course, added to the spectrum of the target to produce the overall spectrum for the mixture. In some instances, depending upon the nature of the drug core and/or the target, one of skill in the art can ascertain by eye peaks corresponding to the drug core in the spectrum of the mixture. This is because the drug cores utilized in this invention have characteristically sharp, narrow peaks, while targets tend to have broader, more diffuse peaks. In such instances there is no need to obtain a spectrum of the target alone at the determined gradient.

In other instances, peaks corresponding to bound drug cores may be obscured by the target spectrum. Therefore, the target spectrum in the absence of drug core needs to be obtained and then subtracted from the mixture spectrum to reveal peaks corresponding to bound drug cores. This may be achieved using standard software utilized in conjunction with NMR techniques.

In a preferred embodiment, the PFG NMR technique is used to quantitate the binding of a drug core to a target. This method comprises the steps of:

i) obtaining one-dimensional NMR spectra of said drug core in the absence of said target at various gradient strengths;

ii) mixing the target with the drug core at a molar ratio of between 1:1 and 1:20.

iii) subjecting said mixture to nuclear magnetic resonance for a period of time sufficient to obtain one-dimensional spectra at the same gradient strengths utilized in step i; and

iv) utilizing the spectral data generated in steps i) and iii) to calculate the Kd between said drug core and said target.

Unlike the line broadening and tNOE methods described above, the PFG method advantageously quantifies the binding of the drug core to the target with more accuracy and substantially less effort. This calculation is achieved by recognizing that rapid exchange of the drug core between the free and bound states leads to an apparent diffusion coefficient Dₐₚₚ:

Dₐₚₚ=(1−p_{b})D_{free}+p_{b}(D_{bound}) [1];

wherein p_{b} is the fraction of ligands that are bound to the target; Dₐₚₚ is the apparent diffusion coefficient of the drug core in the presence of the target; D_{free} is diffusion coefficient of drug core by itself, and D_{bound} is the diffusion coefficient of the bound drug core. The D_{bound} value is the same as the diffusion coefficient of the target, and is readily measured. It should be noted that for higher molecular weight targets, the D_{bound} contribution to equation [1] becomes increasingly negligible. At sufficiently high molecular weight, Dₐₚₚ is well-approximated by the expression (1−p_{b})D_{free}, thus rendering measurement of D_{bound} unnecessary.

Using equation [1], one solves for the bound fraction, p_{b}. Then, from p_{b}, one can calculate K_{d} using the formula:

K_{d}=(Lₜₒₜ/p_{b})×{p_{b}²−p_{b}(1+Pₜₒₜ/Lₜₒₜ)+(Pₜₒₜ/Lₜₒₜ)} [2];

where Lₜₒₜ is the total drug core concentration, and Pₜₒₜ is the total target concentration.

The value of D_{free} is obtained in step i) by performing a fit of the peak height versus gradient strength to an exponential decay function, whose exponent is proportional to the square of the gradient strength. Thus, the choice of gradients at which to generate spectra of the drug core alone must be made so that the peak heights corresponding to the drug core decrease in amplitude as the gradient strength increases. In order to achieve an accurate measurement of D_{free}, it is preferred that between 8 and 16 different gradient strengths be used to generate spectra for the drug core alone.

The values of Dₐₚₚ and D_{bound} are obtained in step iii) by performing a fit of the peak height versus gradient strength to the aforementioned exponential decay function of the ligand (drug core) peaks and resolvable target peaks (usually attributed to aromatic or methyl groups in the target), respectively.

The use of the PFG NMR method to quantify Kd values of any ligand and a target that fall in the micromolar to millimolar range is yet another aspect of the present invention. According to this embodiment, the invention provides a method of quantifying the dissociation constant between a ligand and a target comprising the steps of:

a) obtaining a one-dimensional NMR spectrum of said ligand in the absence of said target at various gradient strengths;

b) mixing the target with the ligand at a molar ratio of between 1:1 and 1:5.

c) subjecting said mixture to nuclear magnetic resonance for a period of time sufficient to obtain one-dimensional spectra at the same gradient strengths utilized in step i; and

d) utilizing the spectral data generated in steps i) and iii) to calculate the Kd between said ligand and said target.

The term “ligand”, as used herein, refers to any molecular entity that is capable of binding to a target. It is preferred that the ligand have a molecular weight of below about 5 kDa. It is even more preferred that the ligand have a molecular weight of below about 2 kDa.

According to another embodiment, the invention provides a drug core library—a plurality of individually compartmentalized compounds and tautomers thereof consisting of:

a) at least one compound of the formula:

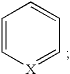

b) at least one compound of the formula:

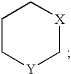

c) at least one compound of the formula:

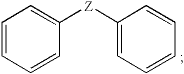

d) at least one compound of the formula:

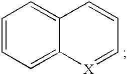

e) at least one compound of the formula:

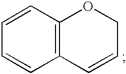

f) at least one compound of the formula:

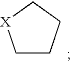

g) at least one compound of the formula:

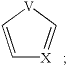

h) at least one compound of the formula:

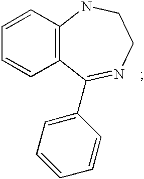

i) at least one compound of the formula:

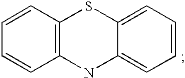

j) at least one compound of the formula:

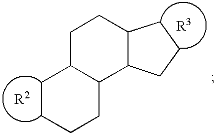

k) at least one compound of the formula:

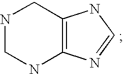

l) at least one compound of the formula:

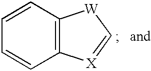

m) at least one compound of the formula:

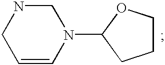

and optionally includes any of:

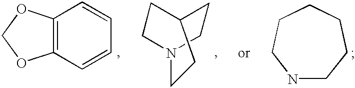

wherein:

V is N or O;

W is N or S;

X is C or N;

Y is C, N or O;

Z is selected from a bond, —CH₂—, —NH—, —O—, —NH—CH₂— or —CH₂—NH—CH₂

R² is a 6-membered carbocyclic ring containing 1, 2 or 3 double bonds

R³, if present, is methylenedioxy; and wherein

any of said compounds is optionally substituted on one or more carbon atoms with one or more substituents independently selected from ═O, ═S, ═N—O—CH₃, —OH, halo, —CN, —(C₁-C₄)-straight or branched alkyl, C₂-alkynyl, —N(R⁴)₂, —C(O)—R⁵, —CH₂C(O)—R⁵, —CH(CH₃)C(O)—R⁵, —OR⁶, —CH₂OH, CH₂NH₂, —CF₃, —S(O) 2NH₂, —S(O)₂OH or —OCH₂CHOHCH₂NH (C₁-C₄) -straight or branched alkyl;

any of said compounds is optionally substituted on one or more nitrogen atoms, if present, with a —(Cₗ-C₃)-straight or branched alkyl or —(CH₂)₁₋₃—OH; and

any of said compounds is optionally substituted on a sulfur atom, if present, with =O; wherein:

each R⁴ is independently selected from H, O, C(O)—CH₃ or —(C₁-C₃)-straight or branched alkyl;

each R⁵ is selected from (CH₂)₀₋₃—OH, O—(C₁-C₃)-straight or branched alkyl, NH₂, or (C₁-C₃)-straight or branched alkyl; and

each R⁶ is selected from —(C₁-C₃)-straight or branched alkyl, or C(O)—(C₁-C₃)-straight or branched alkyl.

More preferably, the plurality of individually compartmentalized compounds consists of:

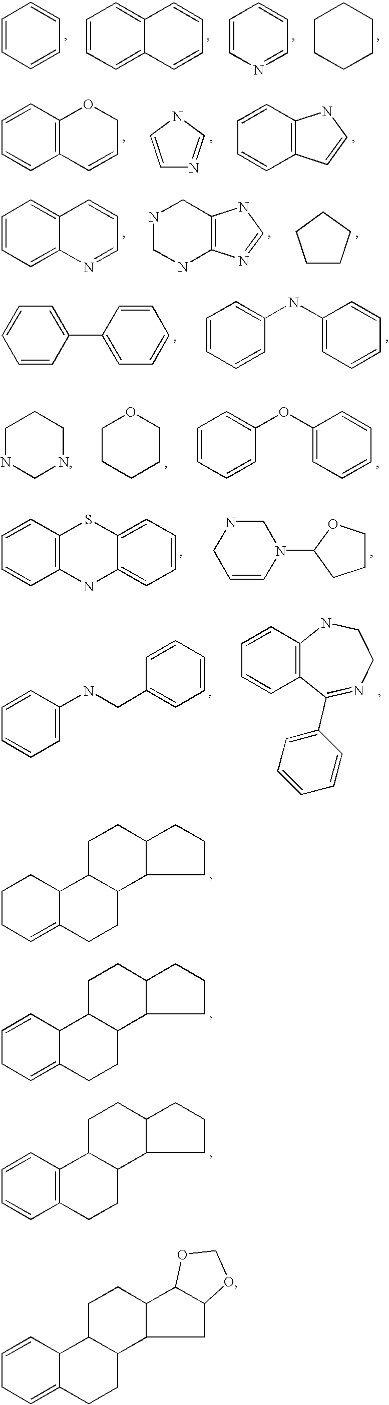

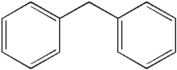

and tautomers thereof; wherein each of said compounds is optionally substituted as set forth above.

According to another preferred embodiment, the optional substituents on one or more carbon atoms are independently selected from ═O, —CH₃, —OH, —OCH₃, —Cl, —NH₂, —C(O)OH, —F, —CH₂OH, —CH₂CH₃, —OC(O)CH₃, —NO₂, —N(CH₃)₂, —CF₃, —C(O)NH₂, —C(O)OCH₃, —C(O)OCH₂CH₃, —CH(CH₃)₂, —S(O)₂NH₂, —C(O)CH₃, —CN, —Br, —I, —S(O)₂OH, —OCH₂CH₃, —CH₂C(O)OH, —OC(O)CH₂CH₃, —CH₂CH(CH₃)₂, —C(O)CH₂OH, —N(H)C(O)CH₃, —C(CH₃)₃, ═S, —CH₂NH₂, —OCH₂CH(OH)CH₂N(H)C(CH₃)₃, —N(H)CH₃, —CH(CH₃)C(O)OH, —C≡CH, —(CH₂)₂CH₃, —CH₂C(O)NH₂, —OCH₂CH(OH)CH₂N(H)CH(CH₃)₂, or ═N—OCH₃;

the optional substituents on one or more nitrogen atoms, if present, are independently selected from —CH₃, —(CH₂)₂OH or —CH₂CH₃; and

the optional substituent on a sulfur atom, if present, is ═O.

Even more preferred is that the substituents attached to a carbon atom are independently selected from ═O, —OCH₃, —OH, —NH₂, —C(O)OH, —S(O)₂OH, —S(O)₂NH₂, —CH₂OH or —C(O)NH₂; and the substituent attached to a nitrogen atom is CH₃.

Preferably, the drug core contains from 0 to 3 total substituents.

The term “individually compartmentalized”, as used herein, refers to each compound being physically separate and apart from one another. The term is intended to encompass each compound being present in a separate container (test tube, vial, or other storage medium commonly used in the art); each compound being attached to a separate bead or other inert, solid media; each compound being present in a separate well in a multi-well plate; and any other means for storing the compounds physically separate from one another.

As discussed in detail, above, the plurality of individually compartmentalized compounds represents moieties that are present in known drugs. Thus, this plurality of compounds represents a small, distinct library of chemical entities each possessing desirable properties for engineering into a drug. These include solubility, bioavailability, ease and low cost of synthesis (including low cost of starting materials and the ability to produce the final product using few synthetic steps), low toxicity, and chemical and metabolic stability.

Because the library of this invention consists of cores and side chains that are present in commercially available drugs, any weak binders detected therein will necessarily have desirable druglike qualities. Moreover, the small size of the library makes screening less time and labor intensive. Finally, the diverse nature of the cores and side chains in the library means that multiple “hits” (i.e., weak binders) allows the flexibility and advantages of pursuing several compound classes at once.

Once a drug core is identified as binding to a target, it can then be modified by any or all of the following: linking with other binding cores, addition of appendages, fusion with other ring structures, addition of substituents and addition of other chemical groups to optimize interaction with the target and produce a drug that is safe and effective to administer to a mammal.

In order that the invention described herein may be more fully understood, the following examples are set forth. It should be understood that these examples are for illustrative purposes only and are not to be construed as limiting this invention in any manner.

### EXAMPLE 1

### Identification of Drug Cores that Bind to p38 MAP Kinase

To demonstrate the feasibility of using DLB and tNOE techniques on a typical drug target, a number of different drug cores were screened against the medium sized protein p38 MAP kinase.

All the spectra were acquired at 5° C. using a Bruker DMX 500 MHz NMR spectrometer. NOESY spectra were collected using 16 transients with 2048 points in ω₂ and 400 points in ω₁ and a mixing time of 400 ms for the drug core mixture in the absence of target and 100 ms for the drug core mixture in the presence of p38. A spin echo with an echo delay of 5 ms was used as a relaxation filter before the t₁ evolution period in all experiments. Samples contained 1 mM of each drug core, 0.2 mM p38 MAP kinase, 25 mM deutero-Tris, 10% (v/v) deutero-glycerol, 20 mM deutero-dithiothreitol at pD*=8.4.

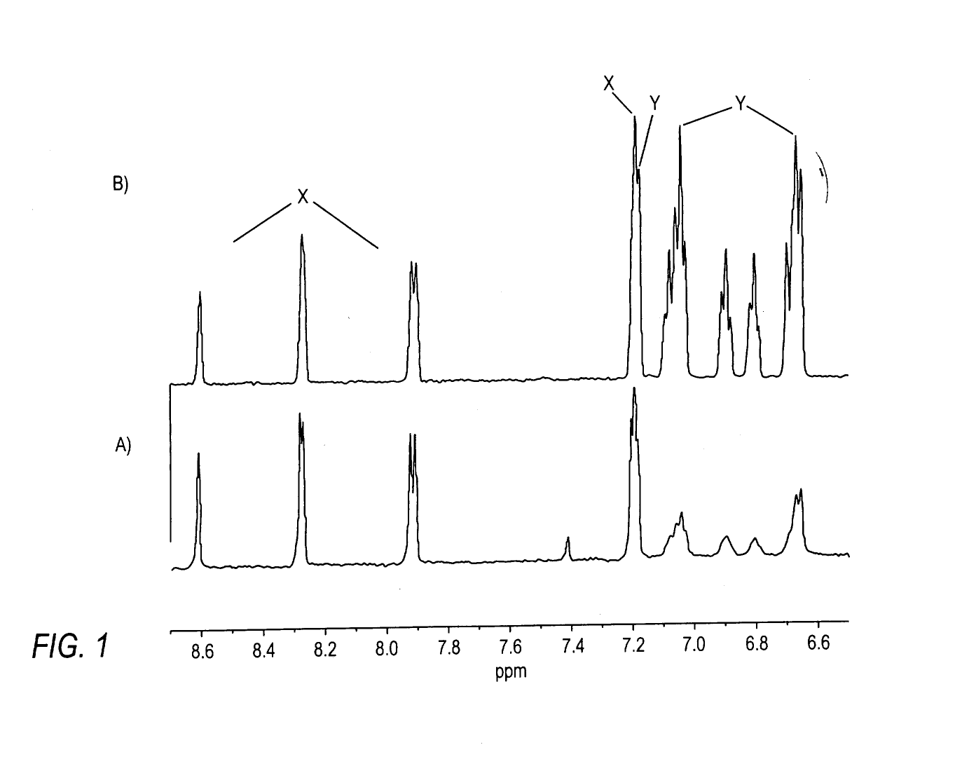

FIG. 1 illustrates line broadening, suppression of fine structure and attenuation of ligand resonance peak height due to the relaxation filter in the presence of the protein, indicating that 2-phenoxy benzoic acid binds to the protein. Close comparison of the two spectra indicate that 2-phenoxy benzoic acid. binds to p38 MAP kinase while nicotinic acid does not.

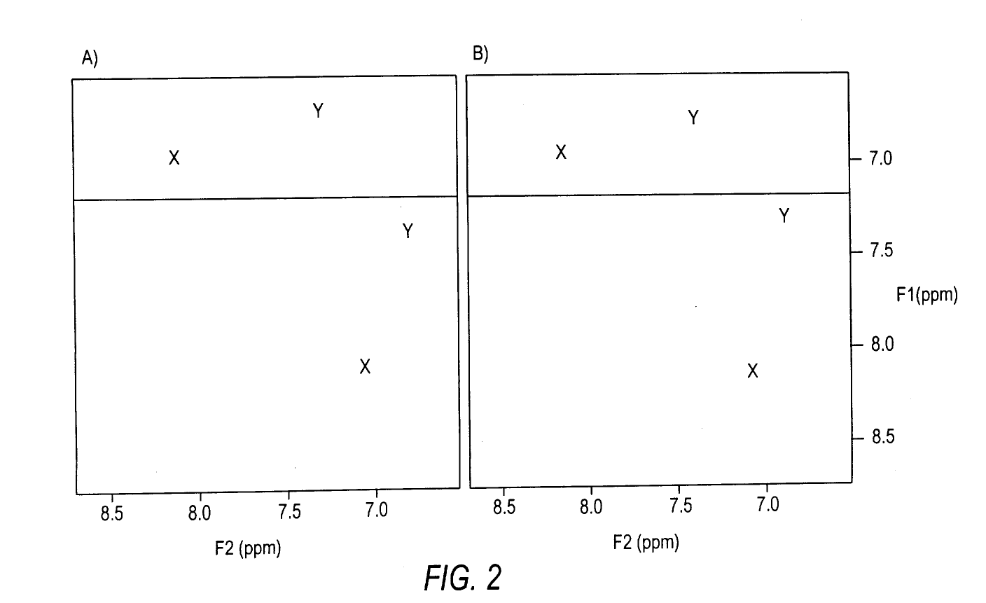

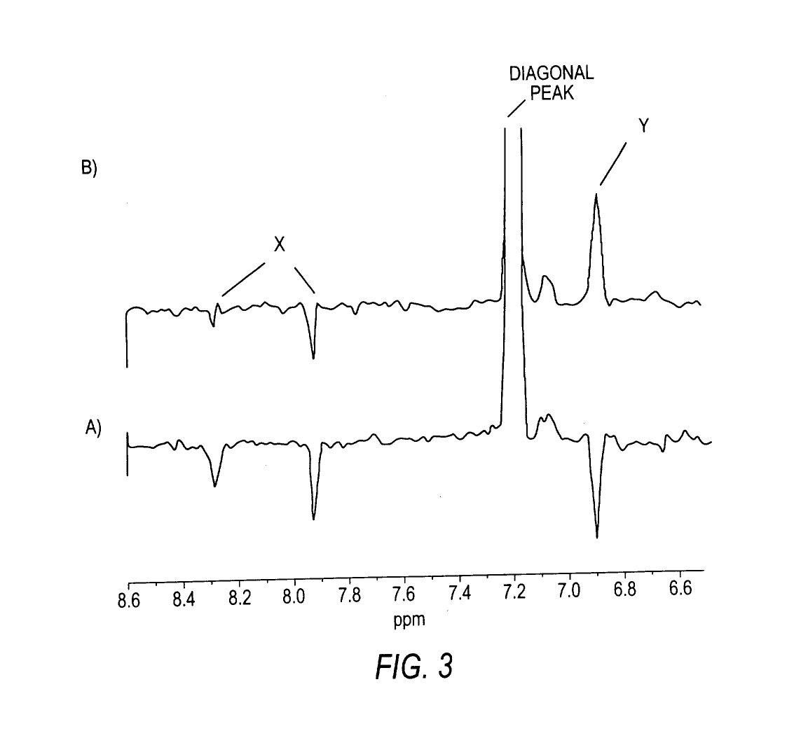

FIG. 2 demonstrates that nicotinic acid (‘X’) and 2-phenoxy benzoic acid (‘Y’) in the mixture without p38 have weak NOE cross peaks, with sign opposite to that of the diagonal peaks (panel A) (this is further illustrated by slices through the 2D spectra shown in FIG. 3). Panel B shows that in the presence of p38, the cross peaks remain opposite in sign from the diagonal peaks for nicotinic acid, indicating this compound does not bind. However, the sign of the cross peaks of 2-phenoxy benzoic are now the same as the diagonal peaks, indicating this compound binds to the protein.

### EXAMPLE 2

### K_{D} Determination of 2-phenoxybenzoic Acid to p38

Two NMR samples were prepared for this study: one sample containing only the 2-phenoxybenzoic acid at 0.5 mM, and another having both p38 and 2-phenoxybenzoic acid at 0.1 mM and 0.2 mM, respectively. The buffer for both samples consisted of 20 mM deutero-DTT, 25 mM deutero-Tris, 10% (v/v) deutero-glycerol. The pD* was set to 8.4. Diffusion coefficients for both the protein and 2-phenoxybenzoic acid were obtained from the p38/2-phenoxybenzoic acid sample, due to the fortuitous appearance of resolved aromatic resonances of p38.

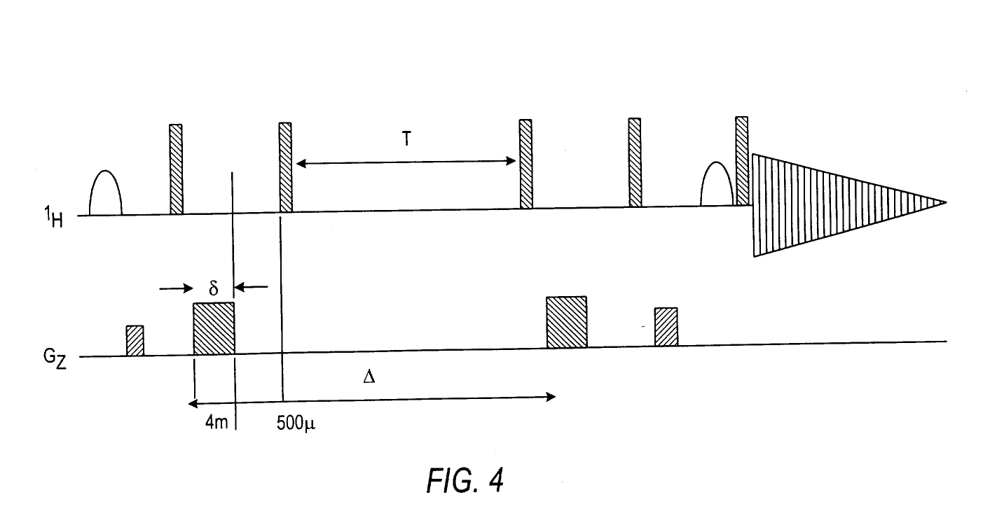

The one-dimensional PFG NMR experiment used for the diffusion measurements was the water-sLED experiment shown in FIG. 4 and described in A. S. Altieri et al., J. Am. Chem. Soc., 117, pp. 7566-7567 (1995), the disclosure of which is herein incorporated by reference. All experiments were carried out at 295K on a Bruker DMX-500 MHz spectrometer. All gradient pulses were rectangular shaped and applied along the z-axis. The critical gradients were the phase-encoding/decoding gradients, corresponding to the two shaded gradient pulses immediately prior and following the “T” period. For both samples, 23 data sets were recorded corresponding to increasing strengths of the phase encoding/decoding gradients. These strengths ranged from 1.3 gauss/cm to 29.4 gauss/cm, in steps of 1.3 gauss/cm.

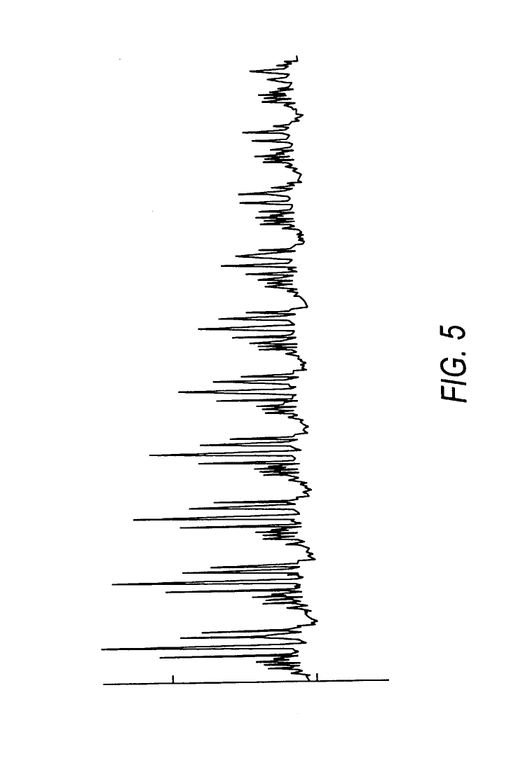

Molecular diffusion between the phase-encoding and phase-decoding gradients attenuated the peak heights of the resulting spectra. This attenuation exacerbates with increasing gradient strength. This effect is illustrated in FIG. 5. Severe peak attenuation occurs for the ligand signals (four larger resonances on the right-hand side), while little attenuation occurs for the p38 aromatic resonances (smaller sharp resonances on the left-hand side).

Denoting a given peak integral by I, the attenuation is described by the decay equation:

I=Aexp{−DK²). [3]

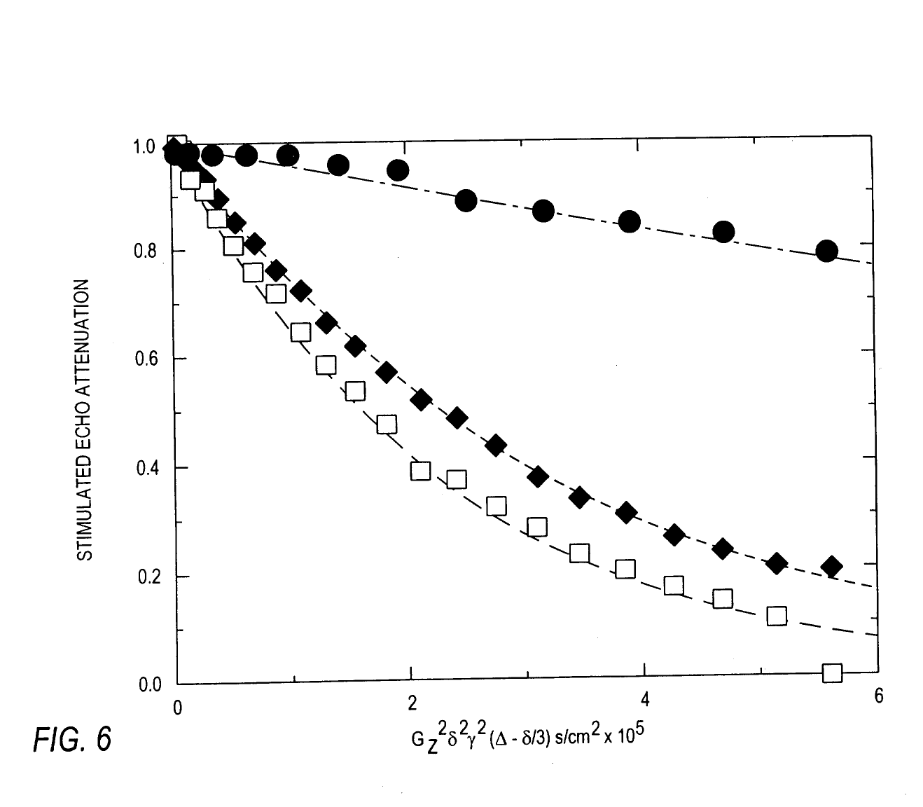

In the above expression, D is the desired diffusion coefficient, A is the peak integral in the absence of the two phase encoding/decoding gradients, and K² is the a factor proportional to the square of the gradient strength given by γ²δ²G_{z}²(Δ−δ/3) Fitting a data file consisting of peak integrals versus K² to equation 3 gives the diffusion coefficient, D, and the prefactor, A. Examples of these fits are shown in FIG. 6.

Data sets for both samples were Fourier-transformed using commercial software (Xwinnmr 1.2, Bruker Instruments, Billerica, Mass.). The resulting peaks were integrated and then listed with the corresponding values for the phase-encoding/decoding gradients. These gradient values were converted into K² values, as defined above in equation 3. Diffusion coefficients were determined by using the well-known Levenburg-Marquardt algorithm to fit the resulting data files to equation 3. The fits are shown in FIG. 6. The 2-phenoxybenzoic acid sample yielded D_{free}=0.438 cm²/sec. The resolved ligand resonances of p38/2-phenoxybenzoic acid sample yielded Dₐₚₚ=0.308 cm²/sec, while the resolved p38 aromatic resonances of the same sample yielded D_{bound}=0.044 cm²/sec.

Inserting these values into equation 1, set forth above, a bound fraction of p_{b}=0.33. Insertion of this p_{b} value into equation 2, set forth above, gave a K_{d} of 70 μM.

While we have hereinbefore presented a number of embodiments of this invention, it is apparent that our basic construction can be altered to provide other embodiments of this invention. Therefore, it will be appreciated that the scope of this invention is to be defined by the claims appended hereto rather than the specific embodiments which have been presented hereinbefore by way of example.

We claim:

1. A method of identifying a drug core suitable for a given target comprising the steps of:
a. providing a drug core consisting of a cyclic structure or a tautomer thereof selected from: 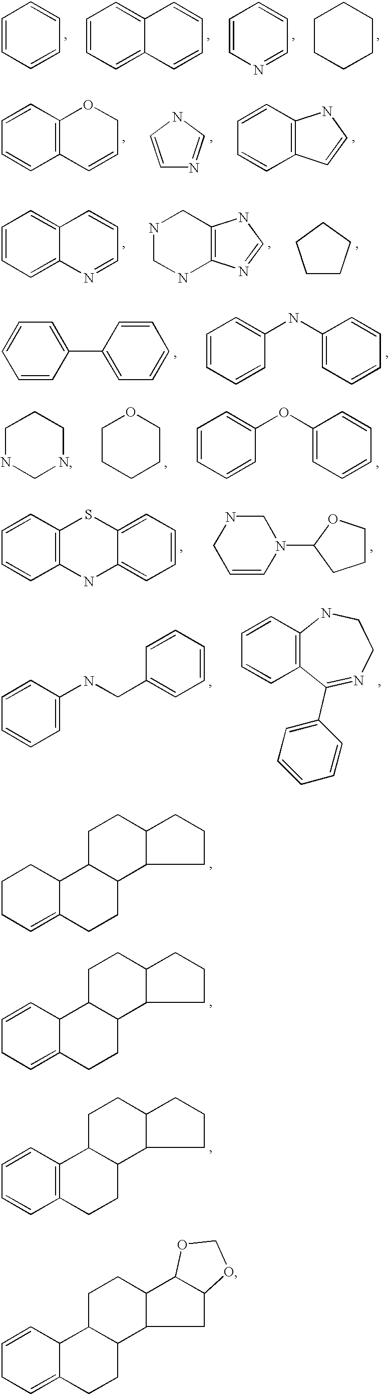 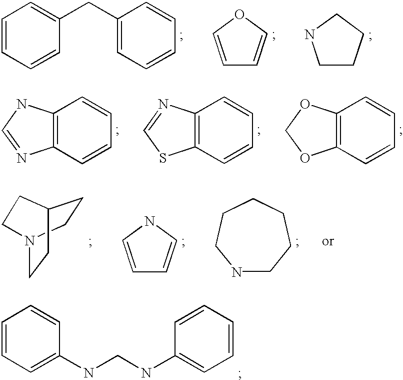
wherein said cyclic structure is optionally substituted at:
i) one or more carbon atoms with one or more substituents independently selected from ═O, —CH₃, —OH, —OCH₃, —Cl, —NH₂, —C(O)OH, —F, —CH₂OH, —CH₂CH₃, —OC(O)CH₃, —NO₂, —N(CH₃)₂, —CF₃, —C(O)NH₂, —C(O)OCH₃, —C(O)OCH₂CH₃, —CH(CH₃)₂, —S(O)₂NH₂, —C(O)CH₃, —CN, —Br, —I, —S(O)₂OH, —OCH₂CH₃₁—CH₂C(O)OH, —OC(O)CH₂CH₃, —CH₂CH(CH₃)₂, —C(O)CH₂OH, —NH—C(O)CH₃, —C(CH₃)₃, ═S, —CH₂NH₂, —OCH₂CHOHCH₂NHC(CH₃)₃, —NHCH₃, —C(CH₃)C(O)OH, —C=—CH, —(CH₂)₂CH₃, —CH₂C(O)NH₂, —OCH₂CHOHCH₂NHCH(CH₃)₂, ═N—OCH₃, or —OCH₂CH₃
ii) one or more nitrogen atoms, if present, with a substituent independently selected from —CH₃, —(CH₂)₂OH or —CH₂CH₃; and
iii) a sulfur atom, if present, with ═O; and
c) determining whether any one of said drug cores binds to said target.

2. The method according to claim 1, wherein: said cyclic structure is selected from: 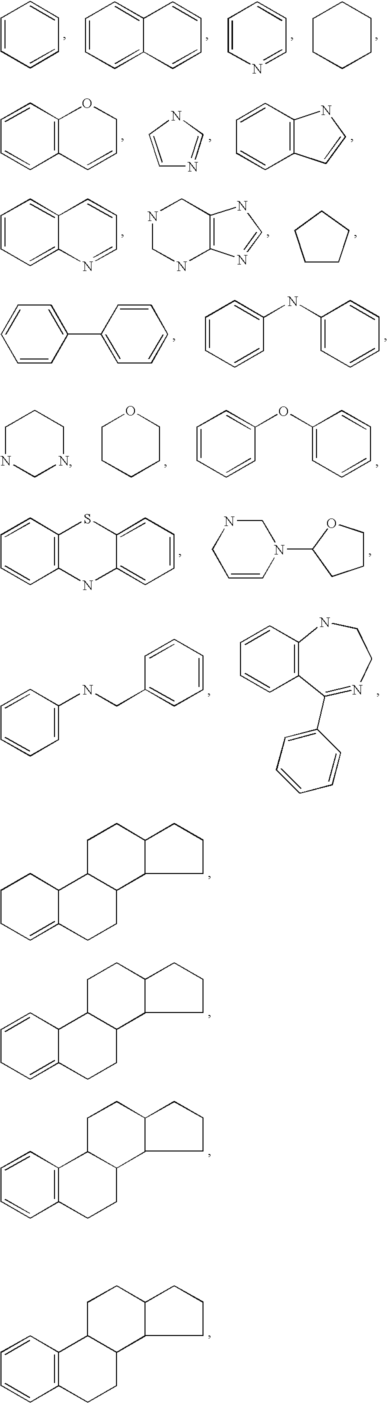 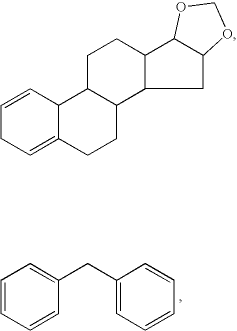
or tautomers thereof; and
said optional substituents on one or more carbon atoms are independently selected from ═O, —CH₃, —OH, —OCH₃, —Cl, —NH₂, —C(O)OH, —F, —CH₂OH, —CH₂CH₃, —OC(O)CH₃, —NO₂, —N(CH₃)₂, —CF₃, —C(O)NH₂, —C(O)OCH₃, —C(O)OCH₂CH₃, —CH(CH₃)₂, —S(O)₂NH₂, —C(O)CH₃, —CN, —Br, —I, —S(O)₂OH, —OCH₂CH₃, —CH₂C(O)OH, OC(O)CH₂CH₃, —CH₂CH(CH₃)₂, —C(O)CH₂OH, —NH—C(O)CH₃, —C(CH₃)₃, ═S, —CH₂NH₂, —OCH₂CHOHCH₂NHC(CH₃)₃, —NHCH₃, —C(CH₃)C(O)OH, —C≡CH, —(CH₂)₂CH₃, —CH₂C(O)NH₂, —OCH₂CHOHCH₂NHCH(CH₃)₂, or ═N—OCH₃.

3. The method according to claim 2, wherein:
said optional substituents on one or more carbon atoms are independently selected from ═O, —OCH₃, —OH, —NH₂, —C(O)OH, —S(O)₂OH, —S(O)₂NH₂, —CH₂OH or —C(O)NH₂; and
said optional substituent attached to a nitrogen atom is CH₃.

4. The method according to any one of claims 1 to 3, wherein determining whether the drug core binds to said target comprises the steps of:
i) obtaining a one-dimensional NMR spectrum of said drug core in the absence of said target;
ii) mixing the target with the drug core at a molar ratio of between 1:1 and 1:100;
iii) subjecting said mixture to nuclear magnetic resonance for a period of time sufficient to obtain a one-dimensional spectrum; and
iv) comparing the spectra obtained in steps i) and iii) to determine if said drug core has bound to said target.

5. The method according to any one of claims 1 to 3, wherein more than one of said drug cores is tested simultaneously for binding to said target; and wherein determining whether the drug core binds to said target comprises the steps of:
i) obtaining one-dimensional NMR spectra for each of said drug cores to be tested for binding to said target, wherein said spectra is obtained in the absence of said target
ii) mixing together between 2 and 20 of said drug cores which will not react with one another;
iii) obtaining a one-dimensional NMR spectrum of said mixture of said drug cores;
iv) mixing said drug cores with the target, wherein each of said drug cores is present at a molar ratio to said target of between 1:1 and 100:1;
v) subjecting said mixture of drug cores and said target to nuclear magnetic resonance for a period of time sufficient to obtain a one-dimensional spectrum; and
vi) comparing the spectra obtained in steps iii) and v) to determine which, if any, of said drug cores has bound to said target.

6. The method according to any one of claims 1 to 3, wherein determining whether the drug core binds to said target comprises the steps of:
i) mixing the target with the drug core at a molar ratio of between 1:1 and 1:100.
ii) subjecting said mixture to nuclear magnetic resonance for a period of time sufficient to obtain a two-dimensional spectrum; and
iii) analyzing the spectrum obtained in step ii) to determine if said drug core has bound to said target.

7. The method according to any one of claims 1 to 3, wherein more than one of said drug cores is tested simultaneously for binding to said target; and wherein determining whether the drug core comprises the steps of:
i) obtaining one-dimensional NMR spectra for each of said drug cores to be tested for binding to said target, wherein said spectra is obtained in the absence of said target
ii) mixing together between 2 and 20 of said drug cores which will not react with one another;
iii) obtaining a one-dimensional NMR spectrum of said mixture of said drug cores;
iv) mixing said drug cores with the target, wherein each of said drug cores is present at a molar ratio to said target of between 1:1 and 100:1;
v) subjecting said mixture of drug cores and said target to nuclear magnetic resonance for a period of time sufficient to obtain a two-dimensional spectrum; and
vi) comparing the spectra obtained in steps iii) and v) to determine which, if any, of said drug cores has bound to said target.

8. The method according to any one of claims 1 to 3, wherein determining whether the drug core binds to said target comprises the steps of:
i) determining a gradient strength that is effective to substantially reduce or eliminate the one-dimensional NMR spectrum of said drug core in the absence of said target;
ii) mixing the target with the drug core at a molar ratio of between 1:1 and 1:20.
iii) subjecting said mixture to nuclear magnetic resonance for a period of time sufficient to obtain one-dimensional spectra using the gradient strength determined in step i); and
iv) analyzing the spectrum obtained in step iii), and, if necessary, comparing said spectrum to a one-dimensional spectrum of said target in the absence of said drug core at the gradient determined in step i), to determine if said drug core has bound to said target.

9. The method according to any one of claims 1 to 3, wherein the determination of whether the drug core binds to said target is quantitative, and comprises the steps of:
i) obtaining one-dimensional NMR spectra of said drug core in the absence of said target at various gradient strengths;
ii) mixing the target with the drug core at a molar ratio of between 1:1 and 1:20.
iii) subjecting said mixture to nuclear magnetic resonance for a period of time sufficient to obtain one-dimensional spectra at the same gradient strengths utilized in step i; and
iv) utilizing the spectral data generated in steps i) and iii) to calculate the Kd between said drug core and said target.

10. A method of calculating the dissociation constant, K_{d}, between a ligand and a target comprising the steps of:
a) obtaining a one-dimensional NMR spectra of said ligand in the absence of said target at various gradient strengths;
b) mixing the target with the ligand at a molar ratio of between 1:1 and 1:20.
c) subjecting said mixture to nuclear magnetic resonance for a period of time sufficient to obtain one-dimensional spectra at the same gradient strengths utilized in step i; and
d) utilizing the spectral data generated in steps i) and iii) to calculate the Kd between said ligand and said target.

11. A plurality of individually compartmentalized compounds consisting of:
a) at least one compound of the formula: 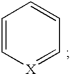
b) at least one compound of the formula: 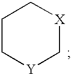
c) at least one compound of the formula: 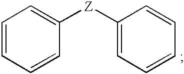
d) at least one compound of the formula: 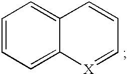
e) at least one compound of the formula: 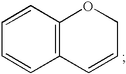
f) at least one compound of the formula: 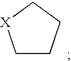
g) at least one compound of the formula: 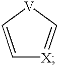
h) at least one compound of the formula: 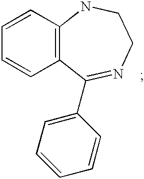
i) at least one compound of the formula: 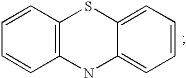
j) at least one compound of the formula: 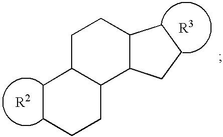
k) at least one compound of the formula: 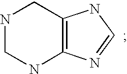
l) at least one compound of the formula: 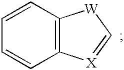
n) at least one compound of the formula: 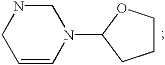
and tautomers thereof; and optionally includes any of: 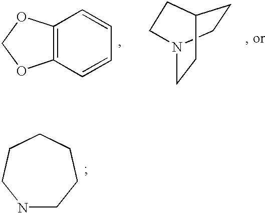
wherein:
V is N or O;
W is N or S;
X is C or N;
Y is C, N or 0;
Z is selected from a bond, —CH₂—, —NH—, —O— or —N—CH₂
R² is a 6-membered carbocyclic ring containing 1, 2 or 3 double bonds
R³, if present, is methylenedioxy; and wherein
any of said compounds is optionally substituted on one or more carbon atoms with one or more substituents independently selected from ═O, —OH, halo, —CN, —(C₁-C₃)-straight or branched alkyl, —N(R⁴)₂, —C(O)—R⁵, —OR⁶, —CH₂H, —CF₃, —S(O)₂NH₂;
any of said compounds is optionally substituted on one or more nitrogen atoms, if present, with a —(C₁-C₃)-straight or branched alkyl; and
any of said compounds is optionally substituted on a sulfur atom, if present, with ═O;
wherein each R⁴ is independently selected from H, O, or —(C₁-C₃)-straight or branched alkyl;
each R⁵ is selected from OH, O—(C₁-C₃)-straight or branched alkyl, NH₂, or (C₁-C₃)-straight or branched alkyl; and
each R⁶ is selected from —(C₁-C₃)-straight or branched alkyl, or C(O)—(C₁-C₃)-straight or branched alkyl.

12. The plurality of individually compartmentalized compounds according to claim 11, wherein said compounds consist of: 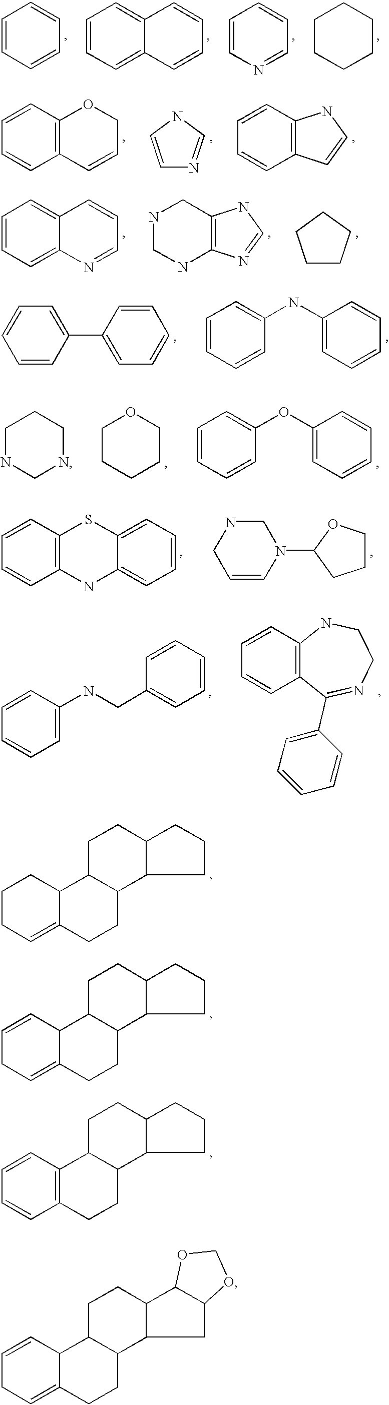 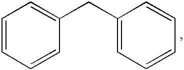
and tautomers thereof; wherein each of said compounds is optionally substituted as set forth in claim 11.

13. The plurality of compounds according to claim 12, wherein:
the optional substituents on one or more carbon atoms are independently selected from ═O, —CH₃, —OH, —OCH₃, —Cl, —NH₂, —C(O)OH, —F, —CH₂OH, —CH₂CH₃, —OC(O)CH₃, —NO₂, —N(CH₃)₂, —CF₃, —C(O)NH₂, —C(O)OCH₃, —C(O)OCH₂CH₃, —CH(CH₃)₂, —S(O)₂NH₂, —C(O)CH₃, —CN, —Br, —I, —S(O)₂OH, OCH₂CH₃, —CH₂C(O)OH, —OC(O)CH₂CH₃, —CH₂CH(CH₃)₂, —C(O)CH₂OH, —N(H)C(O)CH₃, —C(CH₃)₃, ═S, —CH₂NH₂, —OCH₂CH(OH)CH₂N(H)C(CH₃)₃, —N(H)CH₃, —CH(CH₃)C(O)OH, —C≡CH, —(CH₂)₂CH₃, —CH₂C(O)NH₂, —OCH₂CH(OH)CH₂N(H)CH(CH₃)₂, or ═N—OCH₃;
the optional substituents on one or more nitrogen atoms, if present, are independently selected from —CH₃, —(CH₂)₂OH or —CH₂CH₃; and
the optional substituent on a sulfur atom, if present, is ═O.

14. The plurality of compounds according to claim 13, wherein:
the optional substituents attached to a carbon atom are independently selected from ═O, —OCH₃, —OH, —NH₂, —C(O)OH, —S(O)₂OH, —S(O)₂NH₂, —CH₂OH or —C(O)NH₂;
the optional substituent attached to a nitrogen atom is CH₃; and
the optional substituent attached to a sulfur atom is ═O.
